(19) United States
(12) Patent Application Publication
(10) Pub. No.: US 2017/0209301 A1
(43) Pub. Date: Jul. 27, 2017

(54) ENERGY EFFICIENT MANAGEMENT OF HUMAN THERMAL COMFORT

(71) Applicant: Germain DeSeve, Philadelphia, PA (US)

(72) Inventor: Germain DeSeve, Philadelphia, PA (US)

(21) Appl. No.: 15/321,474

(22) PCT Filed: Jul. 7, 2015
(86) PCT No.: PCT/US15/39428

§ 371 Date: Dec. 22, 2016

Related U.S. Application Data
(60) Provisional application No. 62/021,619, filed on Jul. 7, 2014.

(51) Int. Cl. A61F 7/00; A41D 1/00; A61F 7/02; A41D 13/005

(52) U.S. Cl. CPC A61F 7/007; A41D 13/0051; A41D 13/0053; A41D 1/005; A61F 7/02; A61F 2007/0096; A61F 2007/0075; A61F 2007/0093; A61F 2007/0234

(57) ABSTRACT

Embodiments are directed to creating human thermal comfort through energy-efficient management of heat exchangers on areas of the human body that correspond with dermatomes. Embodiments are further directed to a control module that manages a plurality of individual heat exchangers. A physiological state is created that delays or eliminates the onset of uncomfortable thermoregulatory responses to the ambient temperature without attempting to affect the core body temperature, which is applied to garments and other apparatuses to improve human thermal comfort.

### CROSS-REFERENCE TO RELATED APPLICATIONS

This application claims priority to U.S. Provisional Patent Application No. 62/021,619, filed on Jul. 7, 2014 and entitled “Algorithm for Energy Efficient Management of Human Thermal Comfort,” the contents of which are hereby incorporated by reference in their entirety herein.

### BACKGROUND

Products designed to deliver human thermal comfort by physically affecting the core body temperature have been created using liquid cooling and electronics. These products typically enclose the body in a garment or apparatus to establish a comfort envelope.

However, there is a need for a garment and apparatus that is more energy efficient, cost-effective, liquid-free, lighter, and smaller in profile than available products. This need can be met by targeting areas of the body known as dermatomes with very specific thermal inputs in order to create and maintain a physiological sensation of thermal comfort.

### SUMMARY

Embodiments disclosed herein are directed to creating a physiological state of comfort by utilizing heat exchangers on areas of the human body that correspond with dermatomes on the human body. Embodiments are further directed to a control module that manages a plurality of individual heat exchangers.

In one embodiment, a system for providing a sensation of warmth to a user includes: a central processing unit (CPU) for running an algorithm that manages a personal tuning strategy of the user; one or more temperature sensors connected to the CPU and operable to obtain a temperature reading of the user; a plurality of transistor switches, for switching electronic signals, connected to the CPU; a plurality of heat exchange elements capable of conducting sensible heat, each connected to and corresponding to a respective one of the plurality of transistor switches; and a matrix for associating the plurality of heat exchange elements to a plurality of dermatomes of the user, wherein one of the plurality of heat exchange elements corresponds to a respective one or more of the plurality of dermatomes of the user. The CPU obtains a temperature reading from the one or more temperature sensors and, if the temperature reading is not within limits defined by the personal tuning strategy, the CPU implements the algorithm of: turning on each transistor switch, of the plurality of transistor switches, in sequence to deliver power to the corresponding heat exchange element, of the plurality of heat exchange elements in the heat exchanger matrix, for a predetermined period of time to provide a heating thermal sensation to the user at the respective one or more of the plurality of dermatomes; and turning off each transistor switch in sequence. The CPU operates to implement the algorithm until a new temperature reading is within the limits defined by the personal tuning strategy.

In an embodiment, the plurality of heat exchange elements are placed near or adjacent to the user, via the matrix, to correspond with alternating dermatomes, wherein each of the plurality of heat exchange elements is sized to simultaneously address two dermatomes.

In an additional embodiment, the plurality of heat exchange elements and the matrix are incorporated in a garment worn by the user.

A system for providing a sensation of coolness to a user is provided according to another embodiment. In this embodiment, the system includes: a central processing unit (CPU) for running an algorithm that manages a personal tuning strategy of the user; one or more temperature sensors connected to the CPU and operable to obtain a temperature reading of the user; a plurality of transistor switches, for switching electronic signals, connected to the CPU; a plurality of heat stack elements, each connected to and corresponding to a respective one of the plurality of transistor switches, wherein each of the plurality of heat stack elements is comprised of: a thermoelectric (TEM) heat exchanger capable of conducting sensible cooling, a heat sink, and a fan; and a matrix for associating the plurality of heat stack elements to a plurality of dermatomes of the user, wherein one of the plurality of heat stack elements corresponds to a respective one or more of the plurality of dermatomes of the user. The CPU obtains a temperature reading from the one or more temperature sensors and, if the temperature reading is not within limits defined by the personal tuning strategy, the CPU implements the algorithm of: turning on each transistor switch, of the plurality of transistor switches, in sequence to deliver power to the corresponding heat stack element, of the plurality of heat stack elements, for a predetermined period of time to provide a cooling sensation to the user at the respective one or more of the plurality of dermatomes; and turning off each transistor switch in sequence. The CPU further operates to implement the algorithm until a new temperature reading is within the limits defined by the personal tuning strategy.

In an embodiment, the plurality of heat stack elements and the matrix are incorporated in a garment worn by the user.

In an embodiment, the systems may include one or more humidity sensors connected to the CPU and operable to obtain a humidity reading of the user. The CPU implements the algorithm when a humidity reading from the one or more humidity sensors is not within limits defined by the personal tuning strategy, and stops implementing the algorithm when a new humidity reading is within the limits defined by the personal tuning strategy.

In an embodiment, the CPU further operates to control voltage and amperage delivered through the plurality of transistor switches to match a target voltage setting based from the personal tuning strategy.

In an embodiment, the CPU and the plurality of transistor switches are part of a circuit board assembly connectable to a garment worn by the user.

In an embodiment, the personal tuning strategy is inputted to the CPU via an application.

A warming apparatus is provided according to another embodiment. The warming apparatus includes a fabric channel laminated to a matching sheet of adhesive; a layer of garment adhesive laminated to a garment and bonded to the sheet of fabric channel adhesive; a plurality of heat exchange elements capable of conducting sensible heat; a plurality of pieces of reflective insulation, each piece corresponding to one of the plurality of heat exchange elements; and a matrix for associating the plurality of heat exchange elements to a plurality of dermatomes of a user. The plurality of heat exchange elements are wired to a circuit board assembly comprising a central processing unit (CPU) that implements an algorithm of (i) turning on one of the plurality of heat exchange elements in sequence for a predetermined period of time to provide a heating thermal sensation to the user at the related dermatomes based on a sensed temperature reading not within limits defined by the user, (ii) turning off said heat exchange elements in sequence, and (iii) stopping the sequence when a new temperature reading is within the limits defined by the user. The plurality of heat exchange elements and plurality of pieces of reflective insulation are located between the garment adhesive and the fabric channel adhesive.

In an additional embodiment, a cooling apparatus is provided. The cooling apparatus includes: a fabric channel laminated to a matching sheet of adhesive; a layer of garment adhesive laminated to a garment and bonded to the sheet of fabric channel adhesive; a plurality of heat stack elements, each heat stack element comprising: a thermoelectric (TEM) heat exchanger capable of conducting sensible cooling, a heat sink, and a fan; and a matrix for associating the plurality of heat stack elements to a plurality of dermatomes to a plurality of dermatomes of a user. The plurality of heat stack elements are wired to a circuit board assembly comprising a central processing unit (CPU) that implements an algorithm of (i) turning on one of the plurality of heat stack elements in sequence for a predetermined period of time to provide a cooling sensation to the user at the related dermatome based on a sensed temperature reading not within limits defined by a user, (ii) turning off said heat stack elements in sequence, and (iii) stopping the sequence when a new temperature reading is within the limits defined by the user. The plurality of heat stack elements are located between the garment adhesive and the fabric channel adhesive to allow fins of the heat sinks to pass through the fabric channel adhesive and the fabric channel.

In an embodiment, the apparatuses further includes one or more temperature sensors, wherein the CPU obtains a temperature reading from the one or more temperature sensors and determines if the temperature reading is within the limits defined by user.

In an embodiment, one or more of the garment adhesive and the fabric channel adhesive are a thin thermoplastic polyurethane (TPU) adhesive.

In an additional embodiment, a pouch for containing the circuit board assembly is also provided.

### BRIEF DESCRIPTION OF THE DRAWINGS

The foregoing and other aspects of the invention are best understood from the following detailed description when read in connection with the accompanying drawings. The drawings depict embodiments solely for the purpose of illustration; it should be understood, however, that the disclosure is not limited to the specific instrumentalities disclosed. Included in the drawings are the following Figures:

FIG. 1 is a diagram illustrating a dermatome map, which is utilized with embodiments described herein;

FIG. 2 is a diagram illustrating a thermoelectric module (TEM), which is utilized with embodiments described herein;

FIG. 3 is a diagram illustrating Ultra Heating fabric, which is utilized with embodiments described herein;

FIG. 4 is a block diagram illustrating a system for providing a sensation of warmth, according to an embodiment;

FIGS. 5A-5D are exemplary representations of a warming apparatus utilizing the system of FIG. 4, according to an embodiment;

FIG. 6 is a block diagram illustrating a system for providing a sensation of coolness, according to another embodiment;

FIGS. 7A-7D are exemplary representations of a cooling apparatus utilizing the system of FIG. 6, according to an embodiment;

FIGS. 8A and 8B are flowcharts illustrating a method of providing a temperature sensation to a human body, according to an embodiment; and

FIGS. 9A and 9B are research diagrams, illustrating various scientific aspects on which embodiments described herein are based.

### DETAILED DESCRIPTION

Embodiments are directed to creating a physiological state of comfort by utilizing heat exchangers on areas of the human body that correspond with dermatomes on the human body. Embodiments are further directed to a control module that manages a plurality of individual heat exchangers.

According to an embodiment, methods and systems disclosed herein create a physiological state that delays or eliminates the onset of uncomfortable thermoregulatory responses to the ambient temperature without attempting to affect the core body temperature, which is applied to garments and other apparatuses to improve human thermal comfort.

The approach, according to embodiments described herein, utilizes the following principles: use direct thermal conduction through a matrix of heat exchangers to specific dermatomes; at the dermatomes in the matrix, deliver rapidly increasing or decreasing temperatures to the skin that are significantly different from ambient temperature; deliver the thermal conduction for only a short period of time so that energy is not wasted warming or cooling thermoreceptors which are not accepting thermal inputs because of sensory adaptation; change the dermatome being addressed to overcome sensory adaptation and create spatial summation or divergence; and keep the size of the heat exchangers delivering the thermal conduction small to increase efficiency while making the solution as lightweight as possible.

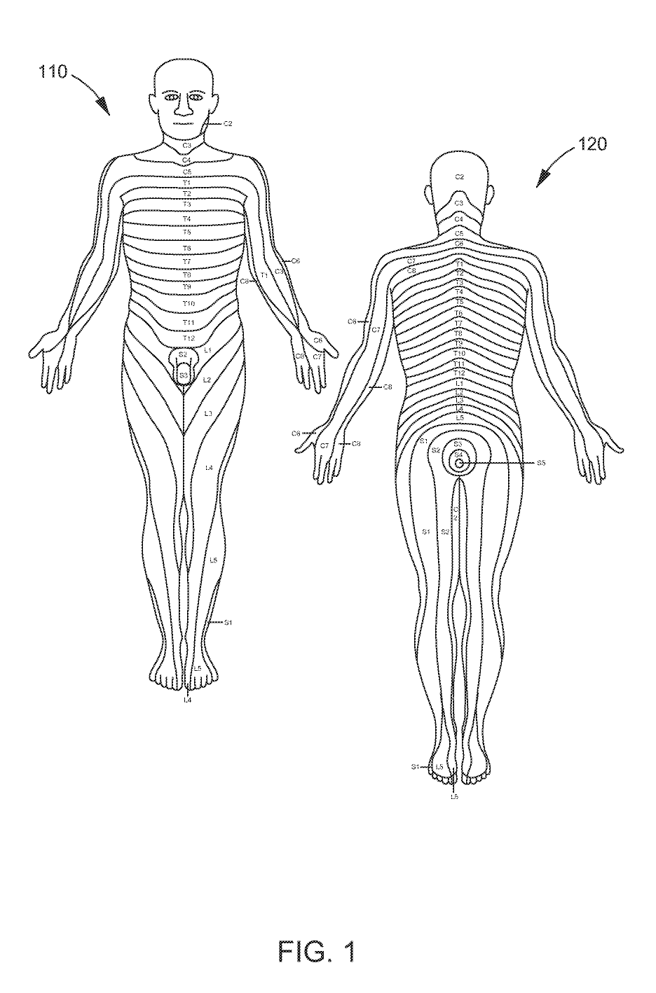

Referring to the drawings, FIG. 1 is a dermatome map 110 (front view), 120 (rear view), which is used with embodiments described herein.

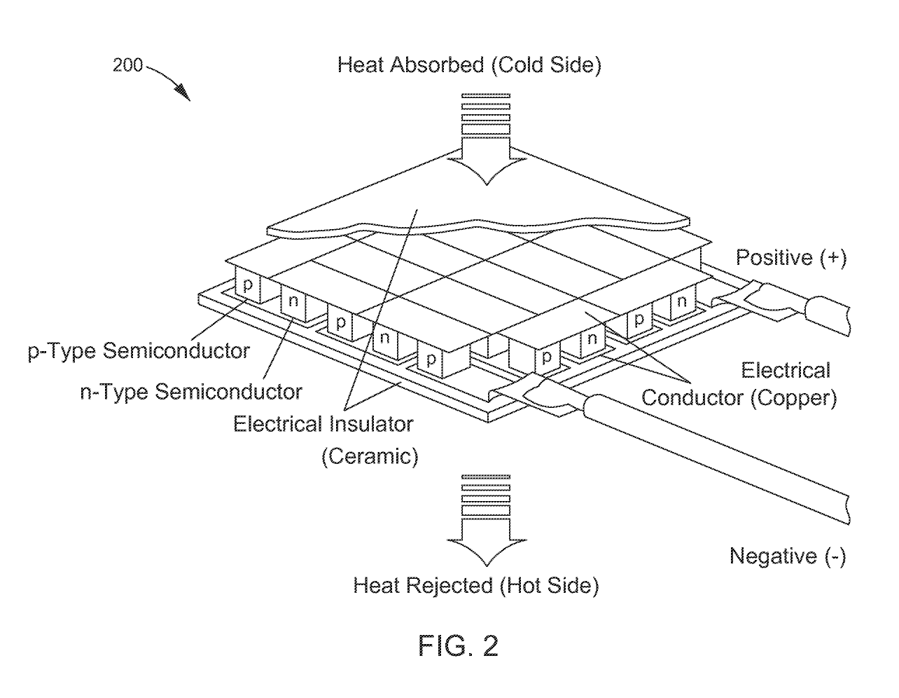

FIG. 2 is a diagram illustrating a standard thermoelectric module (TEM) 200, well known to those of skill in the art, which is utilized with embodiments described herein. The TEM 200 has two sides. When DC current flows through the TEM 200, the TEM creates the the Peltier effect, which brings heat from one side to the other, cooling off one side while the other side gets hotter.

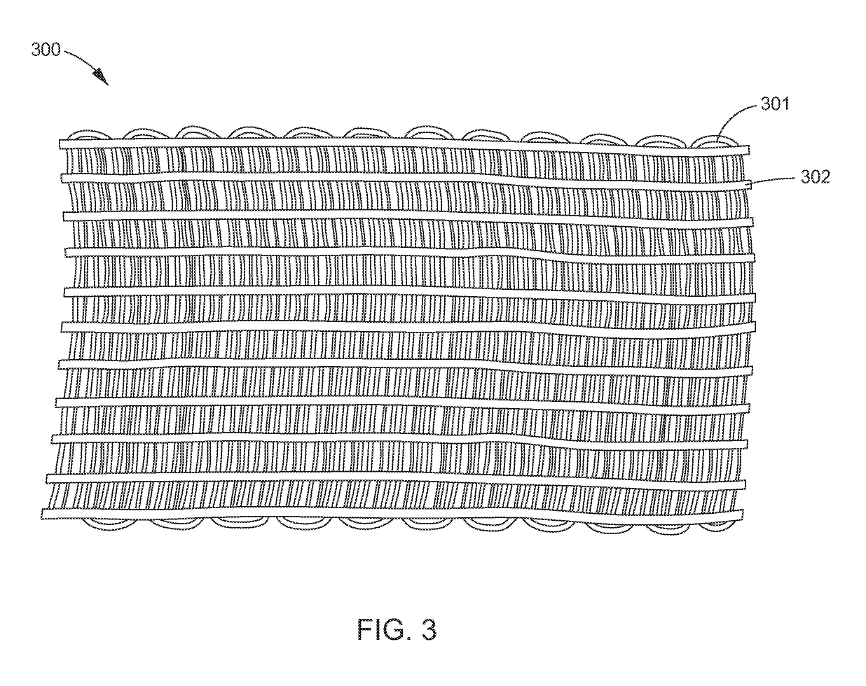

FIG. 3 is a diagram illustrating Ultra Heating Fabric (UHF) 300, which is utilized with embodiments described herein. UHF 300 is, according to an embodiment, a metal-polymer fiber composite conductive yarn heating element 301 in a fabric mesh 302.

There are, as used with embodiments herein, two comfort effects made possible by addressing dermatomes:
1) Addressing adjacent dermatomes with a range of similar warm or cool temperatures creates a convergent physiological effect known as “spatial summation.” Because of spatial summation, an area of the body covered by multiple adjacent dermatomes can feel warm or cool even though heat exchange is not taking place over that entire area, as long as sets of adjacent dermatomes are addressed correctly.
2) Addressing non-adjacent dermatomes with heat exchangers using unexpectedly warm or cool temperatures creates a “divergent” physiological effect: signals from the thermoreceptors sensing the unexpected temperatures will diverge to flood the nervous system, overriding other temperature-related signals. As a result, the brain will focus exclusively on the temperature-related activity at that dermatome and exclude the temperature-related sensations from other dermatomes. This divergent effect is enhanced when the temperature changes rapidly. For example, if an unexpected cold, rapidly falling temperature is applied to a specific dermatome while in a warm room, the brain will not focus on those dermatomes sensing the warm ambient temperature; instead, the changing cold temperature on the specific dermatome will override the brain's processing of sensations of overall warmth.

Each of these effects are used in different embodiments described herein:
1) To create spatial summation, according to an embodiment, a warming apparatus is built using heat exchangers large enough to address adjacent dermatomes simultaneously, and the heat exchangers are spaced in a matrix so that they do not address any other dermatomes. As a result, a user will have an overall sensation of warmth over, for example, eight dermatomes on their trunk, even though only one heat exchanger is warming two dermatomes at any given time during operation.
2) To create a divergent physiological effect, heat exchangers for a cooling apparatus, according to an embodiment, create a change in temperature at a specific dermatome of approximately 14° C. within 10 seconds. In addition, the apparatus is designed so that the heat exchangers are not located on adjacent dermatomes, to prevent spatial summation, which may dull the physiological impact of the temperatures being delivered

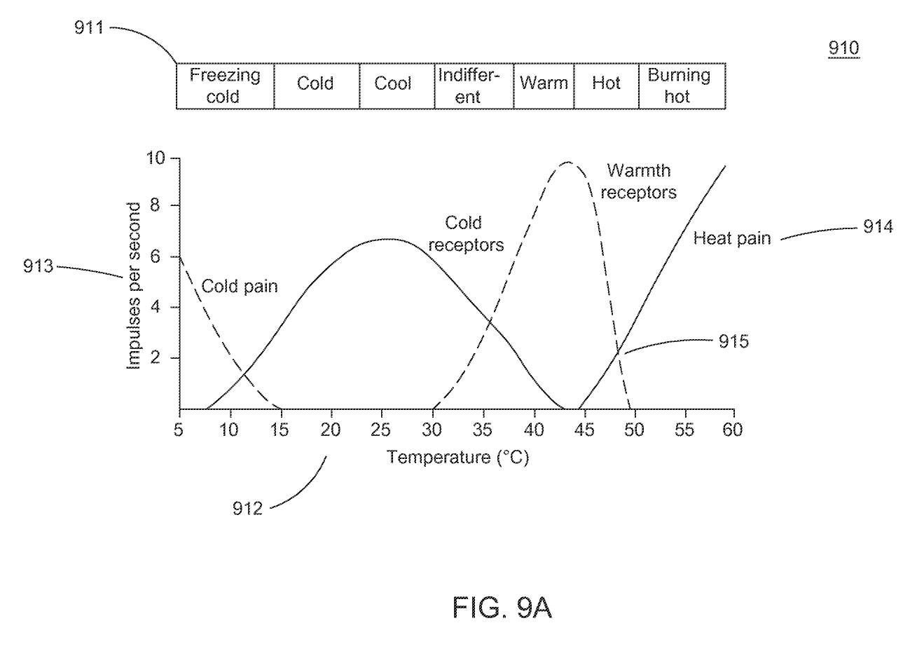

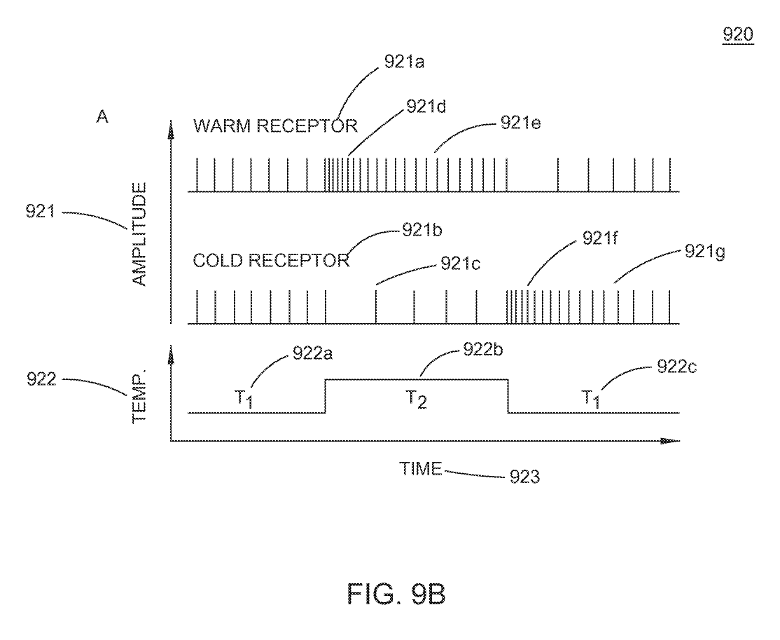

Once these effects have been established, they do not need to be continuously maintained at the same temperature. This is because of the nature of human thermoreceptors, which will only sense temperature for a short duration (approximately 10 seconds) once that temperature has been felt (see FIG. 9B). After the temperature has been felt, the thermoreceptors will not accept similar thermal sensory input for approximately 30 seconds. Because of this natural phenomenon, once the duration for sensing the temperature is over, the heat exchanger for a dermatome can be turned off for 30 seconds. Leaving a heat exchanger on at a dermatome after 10 seconds would be energy inefficient, since that heat exchanger's thermal output will have little to no impact on the user's comfort.

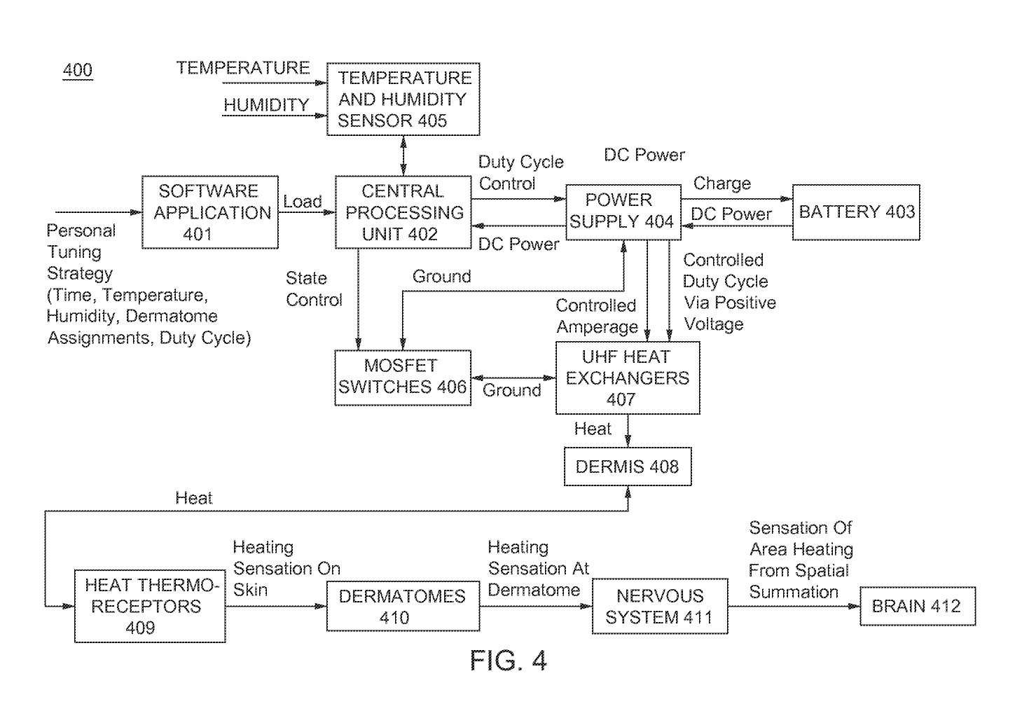

FIG. 4 is a block diagram illustrating a system 400 for providing a sensation of warmth, according to an embodiment.

According to an embodiment, via software application 401, a user uploads a personal tuning strategy to be applied to the system. The personal tuning strategy may comprise various settings, such as, but not limited to, settings for time durations (on state and off state periods), temperature control, humidity control, dermatome assignments, and duty cycle (voltage and amperage control).

When power is applied, the Central Processing Unit (CPU) 402 will start up and run. As it runs, the CPU 402 will apply the personal tuning strategy within the algorithm (described in detail below) as long as the system is powered. Advantageously, there is no need for any input from the user once the user has uploaded their personal tuning strategy: the system runs autonomously.

Typical operation is as follows: A software application 401 is used to load the CPU 402 with the algorithm, described herein, for managing the system components using the personal tuning strategy. This may be done using a USB cable (not shown), and then the USB cable may be removed once loaded.

A power supply 404 may be used to charge a battery 403 using a USB cable and power source (not shown) and then the USB cable may be removed. The battery 403 supplies DC power to the system. The power supply limits power output from the battery 403 to the CPU 402 to 5 Volts and 1 Ampere (Amp).

While the system is running, the CPU 402 will check the temperature provided by the thermistor temperature sensor or temperature and humidity sensor 405. If the temperature is not within the user-tuned parameters, it will turn on each metal-oxide-semiconductor field-effect transistor switch (MOSFET) 406 in sequence for the programmed duration, then turn off the MOSFET 406 until it is turned on again in its sequence. The algorithm will run in a loop until power is interrupted.

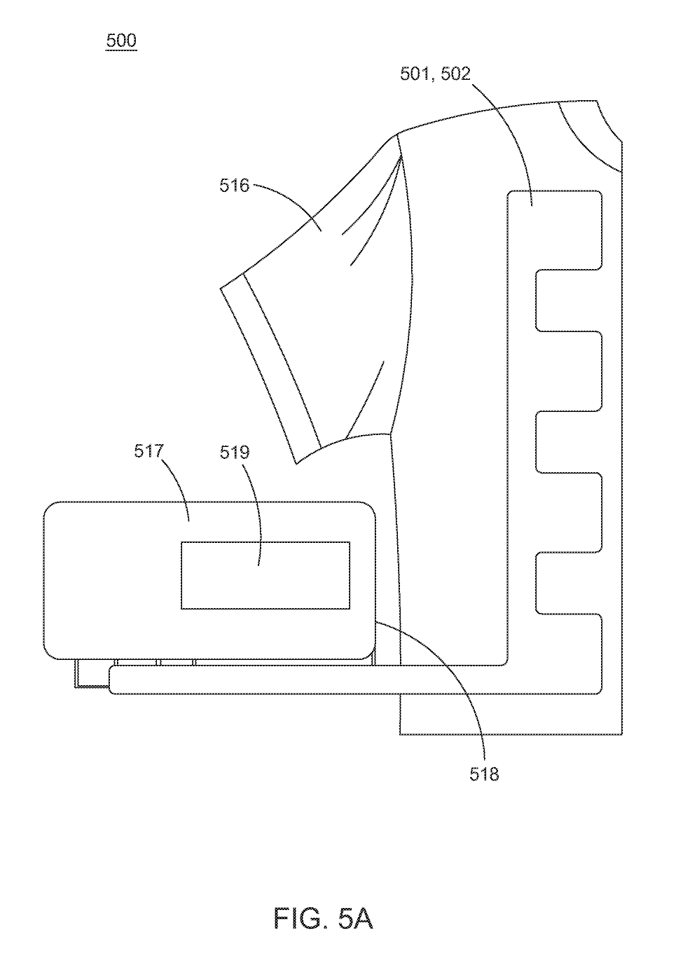

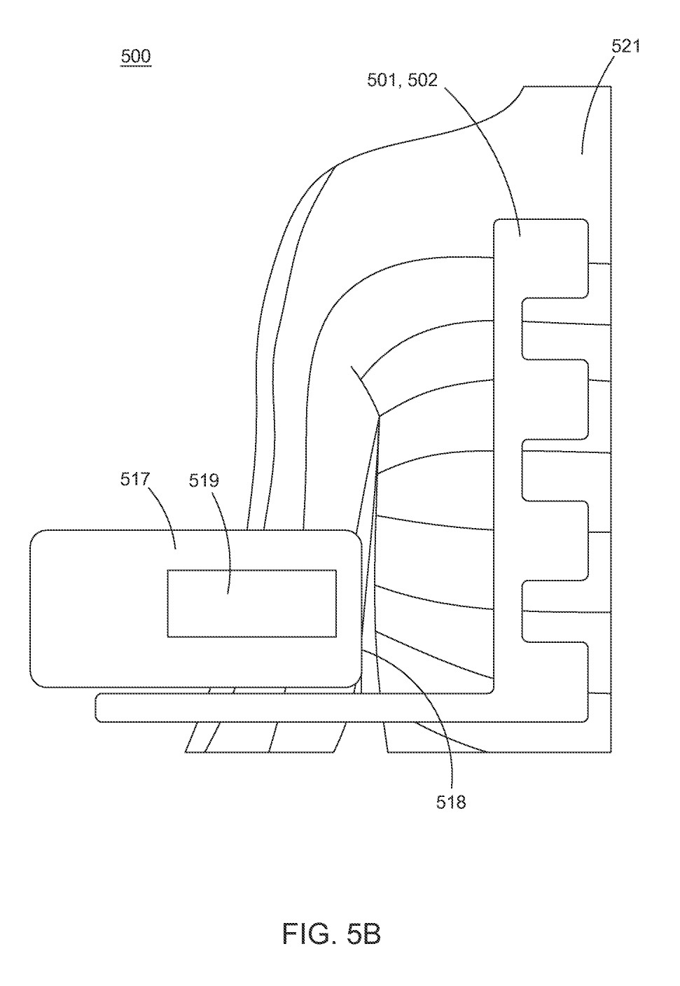

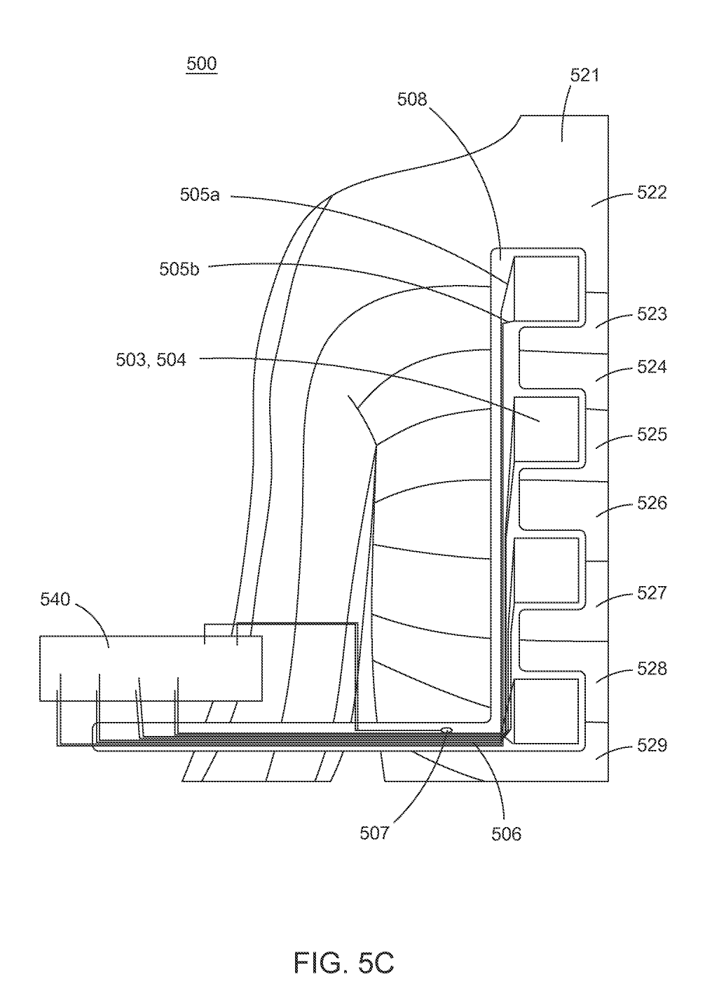

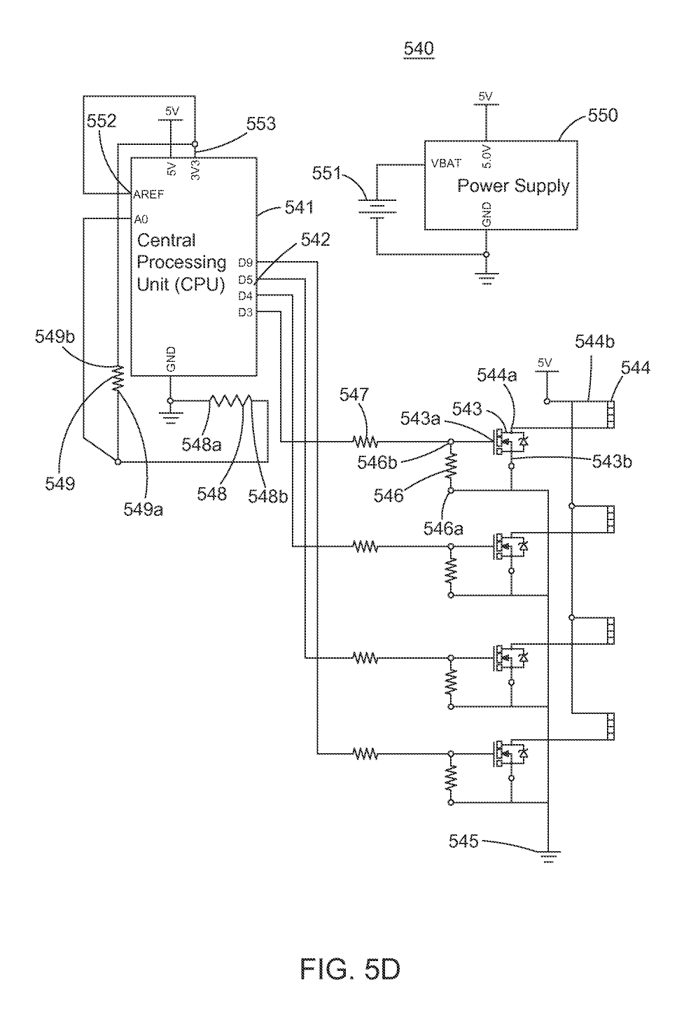

When the MOSFETs 406 are in the on state, the ground circuit is completed between the ground wires of Ultra Heating Fabric (UHF) heat exchangers 407, the MOSFET 406, and the ground circuit on a circuit board containing the system components (see circuit board assembly 540 of FIG. 5D). While the MOSFET 406 is on, power is delivered to the heat exchangers 407. The heat exchangers 407 are arranged in a matrix configuration associated to dermatomes 410 and provide a sensible heating thermal sensation to the user using direct thermal conduction.

As needed, the CPU 402 will also control the voltage and amperage delivered through the MOSFETs 406 while they are in the on state, to match the target voltage setting based from the user's personal tuning strategy.

According to an embodiment, the heat exchangers 407 turn on for ten seconds or less. They heat as quickly as possible, achieving a significant increase in temperature (such as 20 degrees Celsius of heat) at the user's dermis 408. This rapid change in temperature is advantageous because it quickly takes the user's dermis 408 from a cold, cool or indifferent sensation to an indifferent, warm or hot sensation with the change creating the most rapid firing of warmth receptors (see FIG. 9A). The dermis 408 holds the user's heat thermoreceptors 409. The thermoreceptors 409 transmit signals through physiological areas called dermatomes 410. The heating signals travel through their related dermatomes via the nervous system 411 to brain 412. At this point, the brain 412 experiences a sensation of strong heat at the dermatomes 410 being addressed by the heat exchanger 407. Because this strong heating sensation is coming from adjacent dermatomes 410, the brain adds the sensations from the two dermatomes together through a process called spatial summation. The brain also adds the sensations from the pairs of adjacent dermatomes being addressed, creating the physiological effect of spatial summation which is experienced by the nervous system 411 as a sensation of warming over the entire area of the body addressed by the heat exchangers 407, even though only one heat exchanger 407 in the matrix is operating at any given time.

When the MOSFETs 406 are in the off state, the ground circuit is broken between the heat exchangers' 407 ground wires, the MOSFET 406 and the ground circuit on the circuit board. While the MOSFET 406 is off, power is not delivered to the heat exchangers 407.

FIGS. 5A-5D are exemplary representations of the system of FIG. 4 being utilized in a garment, a warming apparatus 500, according to an embodiment. FIG. 5A shows an outer-most view of the warming apparatus 500; FIG. 5B illustrates how portions of the warming apparatus 500 correspond to dermatomes of the human body; FIG. 5C illustrates inner components of the warming apparatus 500; and FIG. 5D is a schematic representation of the warming apparatus 500.

With reference to FIGS. 5A-5D, a fabric channel 501 covers the heat exchanger matrix and some of the system components on a garment 516. The fabric channel is, in an embodiment, laminated to a matching sheet of adhesive 502. In an embodiment, the adhesive 502 may be a thin thermoplastic polyurethane (TPU) adhesive. TPU is a film adhesive that is heat-activated and pressure-activated, and the product used in embodiments herein may have adhesive on both sides. The fabric for the channel 501 may be a typical commercial product, such as, for example, DuPont Lycra. The garment 516 in this embodiment is a typical commercial athletic-type or compression-type shirt, such as a Nike™ Pro Combat shirt. This type of compression shirt is desirable since it holds the heat exchangers close to the body for the maximum conductance of sensible heat. In one embodiment, the garment 516 may be an insulated compression-type garment, which is desirable because it will enable a greater overall sensation of warmth for the user by preventing cold temperatures from reaching the wearer.

In an embodiment, reflective insulation 503 is placed between the adhesive 502 and Ultra Heating fabric (UHF) elements 504. As described above and shown in FIG. 3, UHF is a wire-based resistance heating element in a fabric mesh (see FIG. 3). Each UHF heating element 504 in the heating element matrix is wired for ground 505a and power 505b to a control board (see FIG. 5D) separately using a ribbon cable 506. A 10K Ohm thermistor 507 is placed alongside the ribbon cable 506. In an embodiment, the UHF heating elements 504 have high tensile strength and are lightweight, powerful, and energy efficient.

Reflective insulation 503 provides a radiant barrier that reflects waste heat back towards the garment 516. This improves the energy efficiency of the UHF elements 504 because more heat from the UHF elements 504 s is made into sensible heat, as opposed to being ejected into the external environment.

Adhesive 508 (see FIG. 5A) is laminated onto the garment 516 and is bonded to the to the fabric channel's adhesive 502, with the components 503 (reflective insulation), 504 (UHF elements), 505 (ground and power wiring), 506 (ribbon cable), and 507 (thermistor) between the two adhesive layers 502 and 508. In an embodiment, adhesive 508 is a TPU adhesive.

The bonded adhesives 502 and 508 create a matrix for associating the UHF elements to the dermatomes. The bonding of the two adhesives 502 and 508 creates a waterproof barrier for the components. The fabric channel 501 covers the system components to hold them in place and protect them from damage. The fabric channel 501 also prevents the components from unsafely catching on external objects and/or the user's body parts, etc. In one embodiment, the bonded adhesives are attached to the outside of the garment 516 to hold all components in place on the garment 516. Attaching the components to the outside of the garment 516 minimizes rubbing of the components and fabric channel 501 against the user's skin, which could cause discomfort such as chaffing. The heating element matrix and components are held in place to correctly address the user's dermatomes 521.

The circuit board assembly 540, in an embodiment, is contained in a pouch 517, such as a fabric pouch, that has an opening 518 to receive the circuit board assembly 540, wiring from the thermistor 507, and ribbon cable 506. The pouch 517 may comprise hook-and-loop strips 519 attached to the pouch and one another for securing the pouch 517 and the circuit board assembly 540 to a user. For example, the hook-and-loop strips 519 may be used to secure the pouch 517 on a belt around a user's waist. Other attachment means may alternatively be used, such as, for example, an arm band or direct integration into a garment.

With reference to FIG. 5C, in one embodiment, the UHF elements 504 are used to address four sets of dermatomes 521 as follows, from top to bottom of the UHFs: C5 522 and T1 523; T2 524 and T3 525; T4 526 and T5 527; and T6 528 and T7 529. The area of each UHF element 504 is large enough to address two dermatomes simultaneously. Addressing all of the dermatomes between C5 522 and T7 529 enables the system to deliver a sensation of heat over the entire area between the user's collar and ribcage through spatial summation.

With reference to the schematic of FIG. 5D, a circuit board assembly 540 is illustrated. The central processing unit (CPU) 541 is connected to the circuit board (not shown). Pulse width modulation (PWM) is an industry-standard technique that provides pulsed voltage output. PWM's pulsed output enables continuous operation of an electronic component without providing continuous power. By doing this, PWM reduces energy consumption when compared to continuous consumption. The PWM ports 542 of the CPU 541 are D3, D5, D6 and D9. The CPU 541 is connected, via the CPU's PWM ports 542 using 10K Ohm “pull down” resistors 546, to N-Channel MOSFET transistor switches 543 (MOSFETs) via the MOSFETs' data input 543a. The pull down resistors smooth out the system current to promote long-term reliability of the MOSFETs 543. The MOSFETs 543 are also connected to 220 Ohm resistors 547 to enable the CPU 541 and algorithm to control the system voltage in order to provide consistent voltage to the UHF 544 while each MOSFET 543 is switched on. The MOSFETs 543 are also connected to each UHF 544 via the UHF ground wires 544a, as well as connected 543b to the system ground 545. The MOSFETs 543 are connected 546b to 10K Ohm “pull down” resistors 546 which, in turn, are connected 546a to the system ground 545. Each UHF 544 is connected to the system 5 Volt power 544b.

The thermistor 548 is connected 548b to the CPU 541 on port AO, and to ground 548a, as well as connected 549a to a 10K Ohm resistor 549, and the 10K Ohm resistor 549 is connected 549b to the 3.3V power port of the CPU 553 and the CPU reference signal 752.

System power is sourced through a power supply 550 which steps up the battery voltage to 5 volts (the voltage of the CPU) and provides a regulated 1 Ampere output. The rechargeable battery 551 is connected to the power supply. The CPU measures temperature by measuring the resistance of the thermistor 548 as compared to the reference signal from the CPU 552 and the voltage from the CPU's 3.3 volt output 553.

It is envisioned that other types of heat exchangers could be used, other than the Ultra Heating Fabric (UHF), for example, a carbon wire heating element. It is further envisioned that other arrangements of the UHF heat exchanger matrix 504 may be realized. For example, including more or fewer elements to deliver heat to a larger or smaller number of dermatomes and the related areas of the body. Moreover, it is envisioned that garments other than the garment 516 (i.e., a shirt) may be used; for example, a long-sleeved shirt, pants, leg warmers, arm warmers, shorts, scarf, and essentially any type of clothing that covers more than one dermatome. It is also envisioned that the transistor switches could be other than N-Channel MOSFET switches 543, such as P-Channel MOSFET switches. Additionally, it is envisioned that other arrangements of resistors may be used.

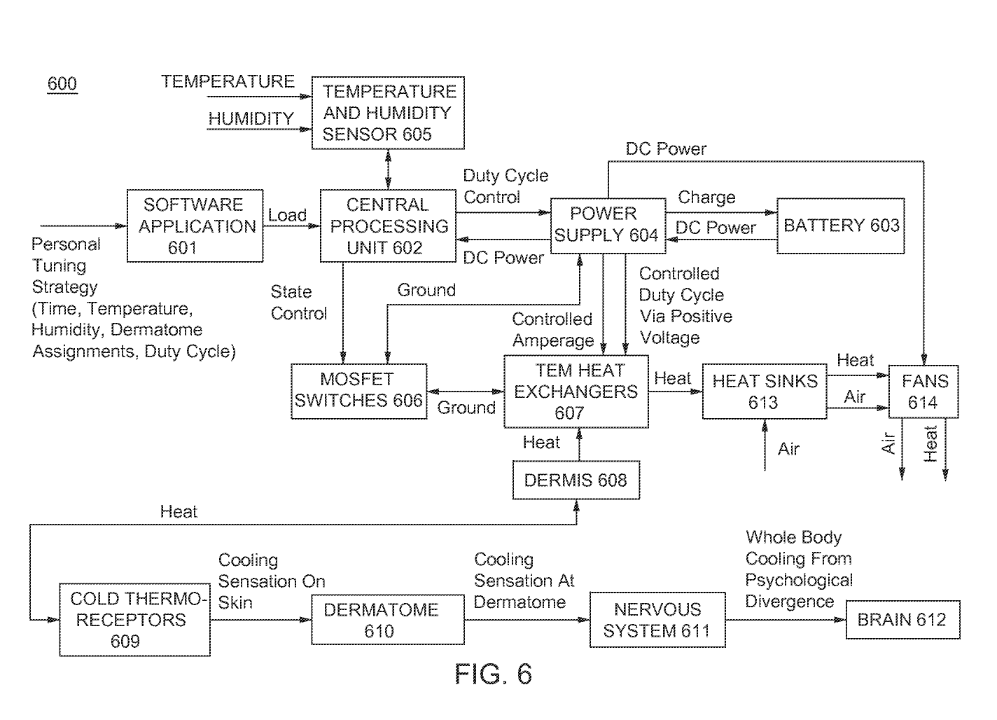

FIG. 6 is a block diagram illustrating a system 600 for providing a sensation of coolness, according to another embodiment.

Similar to the warmth embodiment described above with respect to FIGS. 4 and 5A-5D, according to an embodiment, via software application 601, a user uploads a personal tuning strategy to be applied to the system. The personal tuning strategy may comprise various settings, such as, but not limited to, settings for time durations (on state and off state periods), temperature control, humidity control, dermatome assignments, and duty cycle (voltage and amperage control).

When power is applied, the Central Processing Unit (CPU) 602 will start up and run. As it runs, the CPU 602 will apply the personal tuning strategy within the algorithm (described in detail below) as long as the system is powered. Advantageously, there is no need for any input from the user once the user has uploaded their personal tuning strategy: the system runs autonomously.

Typical operation is as follows: the software application 601 is used to load the CPU 602 with the algorithm (described in detail herein) for managing the system components using the personal tuning strategy. This is done, for example, using a USB cable (not shown), and then the USB cable may be removed.

A power supply 604 is used to charge a battery 603 using a USB cable and power source (not shown), and then the USB cable may be removed. The battery 603 supplies DC power to the system. In an embodiment, the power supply limits power output from the battery 603 to the CPU 601 to 5 Volts and 1 Amp.

While the system is running, the CPU 601 checks the temperature provided by a thermistor temperature sensor or a temperature and humidity sensor 605. If the temperature and/or humidity is not within the user-tuned parameters, the CPU will turn on each MOSFET switch 606 in sequence for the programmed duration, then turn off the MOSFET 606 until it is turned on again in its sequence. The algorithm will run in a loop until power is interrupted.

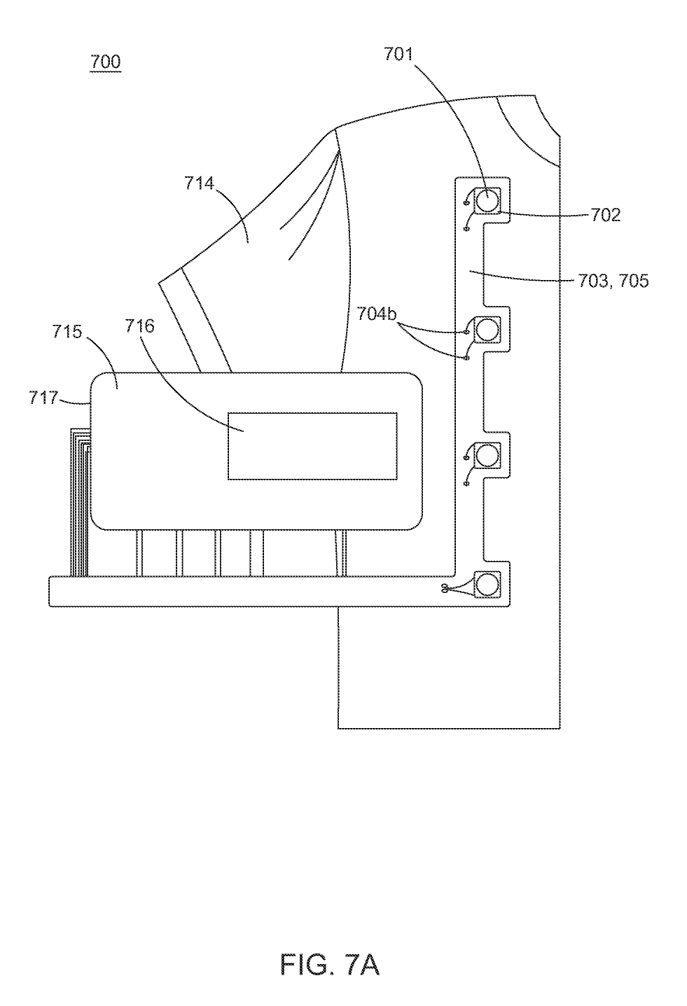

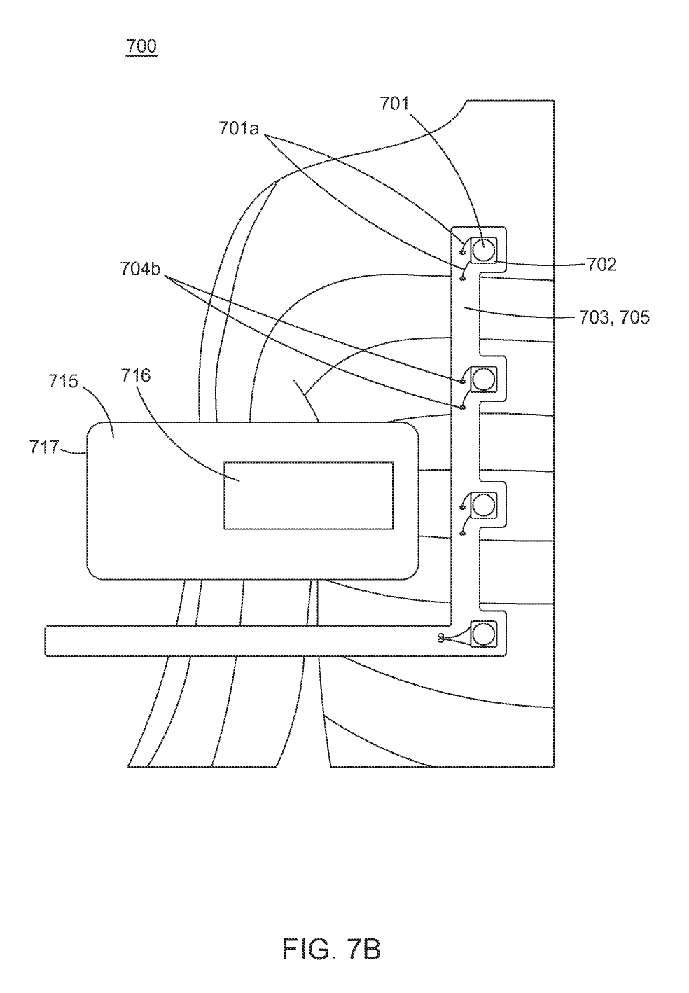

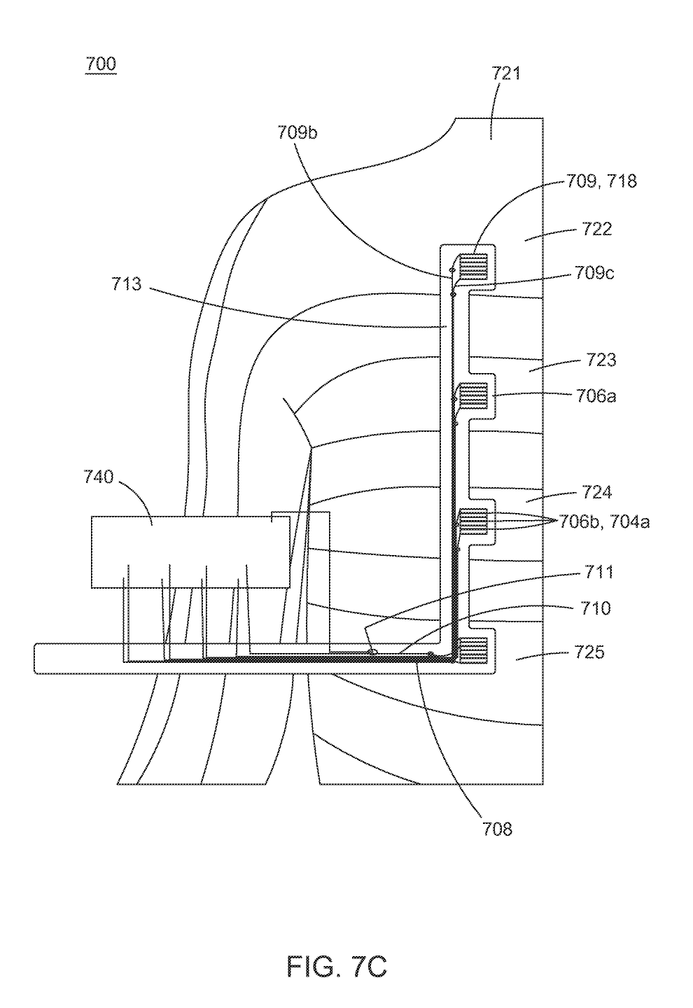

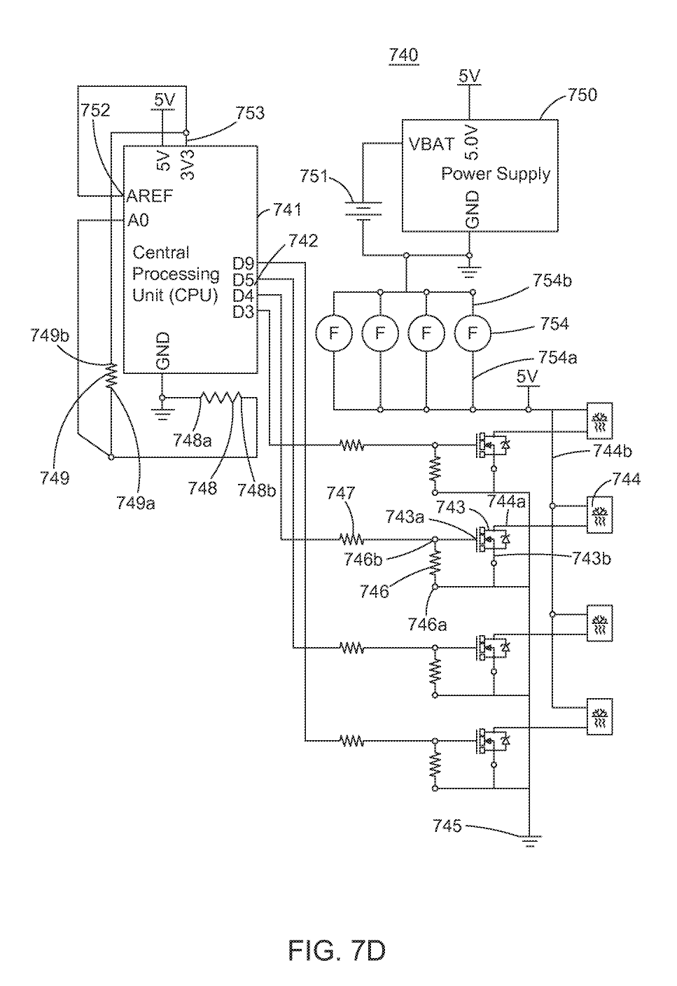

When the MOSFETs 606 are in the on state, the ground circuit is completed between the ground wires of the thermoelectric module (TEM) heat exchangers 607, the MOSFET 606, and the ground circuit on a circuit board containing the system components (see circuit board assembly 740 of FIG. 7D). While the MOSFET 606 is on, power is delivered to the heat exchangers 607 in the matrix of heat exchangers which conduct heat away from the user, thereby delivering sensible cooling to the user. The TEM heat exchangers 607 are placed so that heat is ejected away from the user and toward the external environment, to provide a cooling thermal sensation to the user.

As needed, the CPU 602 will also control the voltage and amperage delivered through the MOSFETs 606 while they are in the on state, to match the target voltage setting based from the user's personal tuning strategy.

In an embodiment, the heat exchangers in the matrix 607 turn on for ten seconds or less. They cool as quickly as possible, achieving a significant decrease in temperature change (such as 14 degrees Celsius of heat removal) at the user's dermis 608. This rapid change in temperature is advantageous because it quickly takes the user's dermis 408 from a hot, warm or indifferent sensation to an indifferent, cool or cold sensation with the change creating the most rapid firing of cold receptors (see FIG. 9A). The dermis 608 holds the user's cold thermoreceptors 609. The thermoreceptors 609 transmit signals through physiological groups called dermatomes 610. The cold signals travel through their related dermatome 610 via the nervous system 611. Because this strong cold sensation is highly out of range when compared to the ambient temperature, and because the sensation is localized to the specific dermatome 610, the cold signals diverge throughout the nervous system 611 and take priority over other competing signals. At this point, the brain 612 receives the signals of strong cold at the dermatome 610 being addressed by the heat exchanger 607 and excludes other thermal signals. Since the brain 612 primarily receives thermal sensations of strong cold, there is an overall sensation of the entire body being cool. This sensation lasts until sensory adaptation has taken place at the cold thermoreceptors 609 at the affected dermatome, a period of about 10 seconds (see FIG. 9B). Cold thermoreceptors 609 at the affected dermatome 610 will not be able to experience a strong cold sensation again until approximately 20-30 seconds have elapsed.

The thermoelectric module (TEM) heat exchangers 607 eject heat to heat sinks 613 via conduction.

Air is moved through the heat sinks to promote conductive-convective heat transfer by fans 614. The fans 614 operate while the system 650 is powered. The heated air from the fans 614 is ejected into the outside environment.

When the MOSFETs 606 are in the off state, the ground circuit is broken between the heat exchangers' 607 ground wires, the MOSFET 606, and the ground circuit on the circuit board assembly 740 (again, see FIG. 7D). While the MOSFET 606 is off, power is not delivered to the matrix of heat exchangers 607. The fans 614 continue to operate while the system 650 is powered.

FIGS. 7A-7D are exemplary representations of the system of FIG. 6 being utilized in a garment, a cooling apparatus 700, according to an embodiment. FIG. 7A shows an outer-most view of the cooling apparatus 700; FIG. 7B illustrates how portions of the cooling apparatus 700 correspond to dermatomes of the human body; FIG. 7C illustrates inner components of the cooling apparatus 700; and FIG. 7D is a schematic representation of the cooling apparatus 700.

With reference to FIGS. 7A-7D, a plurality of fans 701 are attached to respective ones of a plurality of heat sinks 706a. In an embodiment, an adhesive 702 may be used to attach the fans 701 to the heat sinks 706a. The adhesive may be, for example, a typical commercial tape. The heat sinks 706a may be typical commercial finned aluminum units.

A fabric channel 703 covers the heat exchanger matrix and components on a garment 714. The fabric for the channel 703, in an embodiment, is a typical commercial product, such as DuPont Lycra. The garment 714 in this embodiment is a typical commercial athletic-type or compression-type shirt, such as the Compression version of a Nike Pro Combat shirt. This type of compression shirt is desirable since it holds the heat exchangers close to the body for the maximum cooling sensation. The fabric channel 703 has openings 704b for the wires 701a from the fans 701. The fabric channel 703 also has openings 704a that correspond to fins 706b of the heat sinks 706a (see FIG. 7C). Strips of the laminated fabric from the fabric channel 703 lie between the heat sink fins 706b securely hold the heat sink 706a and the other components attached to it in place in alignment on the fabric channel 703.

The fabric channel 703 may be, in an embodiment, laminated to a matching sheet of adhesive 705, such as TPU adhesive that is heat-activated and pressure-activated. The adhesive 705 is cut to allow the fins 706b of the heat sinks 706a to pass through the TPU sheet and the fabric channel 703. The adhesive 705 is also cut 704b to allow the wires of the fans 701 to pass through the adhesive 705 and the fabric channel 703. The fabric channel 703 covers the system components to hold them in place and protect them from damage. The fabric channel 703 also prevents the components from unsafely catching on external objects and/or the user's body parts, etc.

The heat sinks 706a are attached to the Thermoelectric Modules (TEMs) 709 using, for example, thermally conductive tape 718. The heat sinks 706a may be typical commercially available aluminum units. The TEMs 709 in this embodiment are commercially available units. The thermally conductive tape may be a typical commercial variety.

The wires 701a of the fans 701 are connected to the circuit board assembly 740 (see schematic diagram of FIG. 7D) using a ribbon cable 708. The fans may be typical commercially available units.

Each TEM 709 is wired for ground 709b and power 709c to the circuit board assembly 740 separately using a ribbon cable 710. A 10K Ohm thermistor 711 is placed alongside the ribbon cable 710.

In an embodiment, each TEM 709 may be held by thermally conductive adhesive 712 to a sheet of adhesive 713, such as TPU adhesive, that matches the fabric channel adhesive 705. The adhesive 713 is laminated onto the garment 714 and is bonded to the fabric channel's adhesive 705, with the components placed between the two adhesive layers 705 and 713. The bonded adhesives 705 and 713 create a matrix for associating the TEM elements to the dermatomes.

In an embodiment, the TEMs 709 are placed so their cooling effect will be directed toward the garment 714 and its user. This effect creates heat on the side of the TEM 709 facing away from the garment 714.

According to an embodiment, the heat exchanger stack (“HE stack”) is designed so that heat removed from the TEM 709 is effectively ejected into the external environment. The HE stack is comprised of the following components, which move heat from the garment 714 to the external environment while holding the stack together and in place:
a) The thermally conductive adhesive 712 for attaching the garment 714 via the adhesive sheet 713 to the matrix of TEMs 709;
b) The matrix of TEM heat exchangers 709;
c) The thermally conductive adhesive 718 for attaching the TEMs 709 to the heat sinks 706a;
d) The heat sinks 706a;
e) The heat sink-fan adhesive 702; and
f) The fans 701.

The thermally conductive adhesive 712 for attaching the garment 714 to the TEMs 709 holds the components together while conducting temperature between them. When electric current is passed through the TEMs 709, they exchange heat by moving it from one side of the TEM's surface to the other side (see FIG. 2). The TEMs 709 used in this embodiment are rugged enough for use in a garment, as well as lightweight, powerful, small, and energy efficient.

The thermally conductive adhesive for attaching the TEMs 709 to the heat sinks 706a holds the components together while conducting temperature between them.

The aluminum in the heat sinks 706a ejects heat away from the TEMs 709 by conducting it and spreading it over the surface area of the heat sink 706a, which is significantly larger than the surface area of the TEM 709. The larger surface area provided by the heat sink 706a promotes efficient and effective heat exchange between the TEM 709, and the fans 701, and ultimately the external environment.

When power passes through the fans 701, they spin and increase the airflow through the heat sinks 706a. This significantly improves the heat ejection capabilities of the HE stack when compared to using TEMs and heat sinks without fans and prevents the TEMs from overheating, which can lead to them failing.

With reference to the schematic of FIG. 7D, a circuit board assembly 740 is illustrated. The central processing unit (CPU) 741 is connected to the circuit board (not shown). PWM is an industry-standard technique that provides pulsed voltage output. PWM's pulsed output enables continuous operation of an electronic component without providing continuous power. By doing this, PWM reduces energy consumption when compared to continuous consumption. The PWM ports 742 of the CPU 741 are D3, D5, D6 and D9. The CPU 741 is connected via the CPU's PWM ports 742 using 10K Ohm “pull down” resistors 746 to N-Channel MOSFET transistor switches 743 (MOSFETs) via the MOSFETs' data input 743a. The pull down resistors 746 smooth out the system current to promote long-term reliability of the MOSFETs 743. The MOSFETs 743 are also connected 746b to 220 Ohm resistors 747 to enable the CPU 741 and algorithm to control the system voltage in order to provide consistent voltage to the TEM 744 while each MOSFET 743 is switched on. The MOSFETs 743 are also connected 744a to each TEM 744 via the TEM ground wires, as well as connected 743b to the system ground 745. The 10K Ohm resistors 746 are connected 746a to the system ground 745. Each TEM 744 is connected to the system power 744b.

The thermistor 748 is connected 748b to the CPU on port AO, and to ground 748a, as well as connected 749a to a 10K Ohm resistor 749 and that resistor is connected 749b to the 3.3 volt power port of the CPU 753 and the CPU reference signal 752.

In an embodiment, system power is sourced through power supply board 750 which steps up the battery voltage to 5 Volts (the voltage of the CPU) and provides a regulated 1 Ampere output. The rechargeable battery 751 is connected to the power supply board 750. The CPU 741 measures temperature by measuring the resistance of the thermistor 748 as compared to the CPU reference signal 752 and voltage from the 3.3 volt output of the CPU 753.

Four fans 754 are connected to the system power 754a and ground 754b.

The circuit board assembly 740, in an embodiment, is contained in a pouch 715, such as a fabric pouch, that has an opening 717 to receive the circuit board assembly 740, wiring from the thermistor 711, and ribbon cables 708 and 710. The pouch 715 may comprise hook-and-loop strips 716 attached to the pouch and one another for securing the pouch 715 and the circuit board assembly 740 to a user. For example, the hook-and-loop strips 716 may be used to secure the pouch 715 on a belt around a user's waist. Other attachment means may alternatively be used, such as, for example, an arm band or direct integration into a garment.

In one embodiment, the TEMs 709 are used to address four dermatomes 721 as follows, from top to bottom of the TEMs 709: C5 722; T2 723; T4 724; and T6 725. The area of each TEM 709 is small enough to address a specific dermatome 721. The dermatomes 721 in the design are chosen because they are non-adjacent. This design means possible negative effects from spatial summation are minimized.

It is envisioned that other heat exchangers, other than thermoelectric modules (TEMs) could be used, such as an electric resistance heating element. It is further envisioned that other arrangements of the TEMs 709 may be realized; for example, including more or fewer elements. Moreover, it is envisioned that garments other than the garment 714 (i.e., a shirt) may be used; for example, a long-sleeved shirt, pants, leg warmers, arm warmers, shorts, scarf, and essentially any type of clothing that covers more than one dermatome. It is also envisioned that the transistor switches could be other than N-Channel MOSFET switches 743, such as P-Channel MOSFET switches. Additionally, it is envisioned that other arrangements of resistors could be used. It is also envisioned that other arrangement of the components can be used, such as a side-by-side arrangement instead of a stacked arrangement.

According to an embodiment, both the warming and the cooling apparatuses 500, 700 may be managed by a user. The user is able to change the system's settings to adjust it to meet their personal comfort needs. This is done by creating “personal tuning strategies” that inform the system about how to deliver comfort to the user. The user-tunable system settings may include, but are not limited to, time, temperature, humidity, voltage, and amperage.

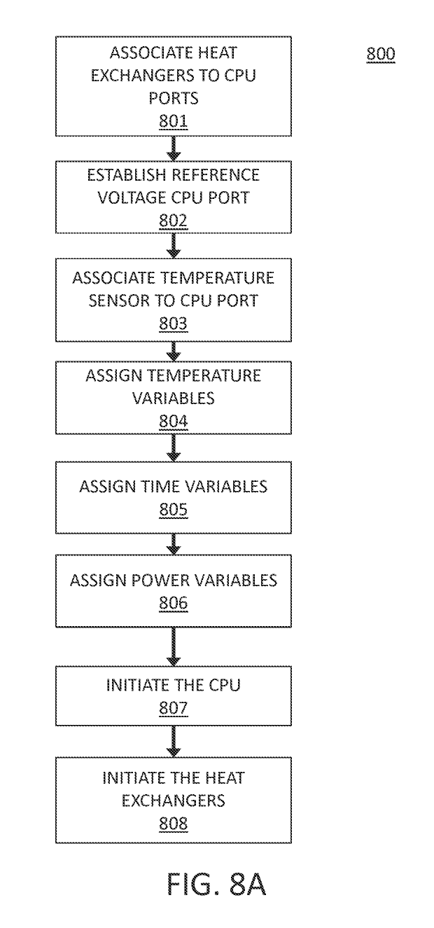

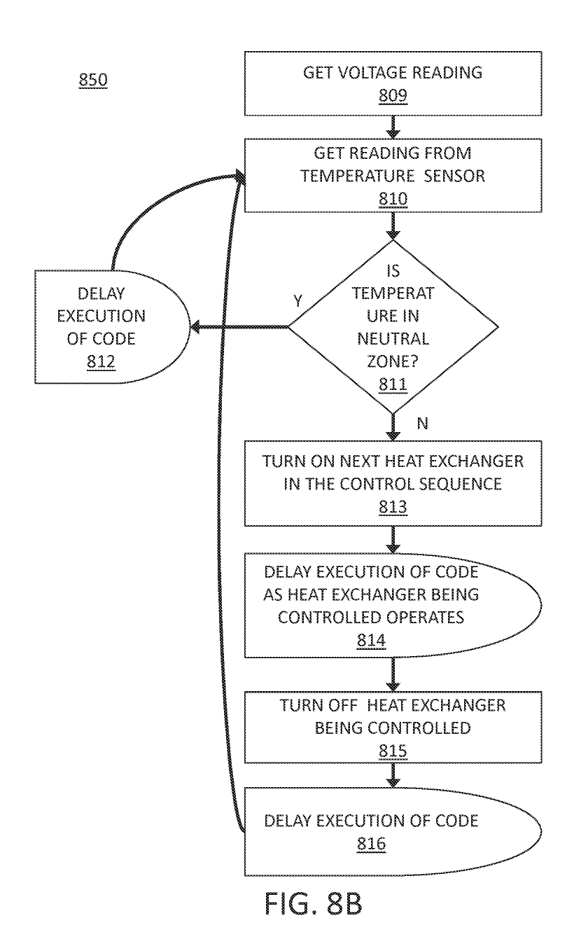

FIGS. 8A and 8B are flowcharts illustrating a method of providing a temperature sensation to a human body, according to an embodiment.

First with reference to FIG. 8A, at 801, the system identifies a plurality of solid state heat exchangers (HEs) and assigns them to ports (also known as “pins”) on a computer with memory storage and a microprocessor (central processing unit or “CPU”). In an embodiment, four switched HEs are identified and assigned to CPU ports. At 802, a CPU port for reference voltage is assigned. The output from the CPU reference voltage port will be used to ensure that the HEs do not overheat or overcool by consuming too much power, or underheat or undercool if the actual voltage being delivered by the system is lower than the system's rated voltage. At 803, temperature sensors are identified and associated with a port on the CPU. At 804, temperature variables that define the bounds of the thermal “neutral zone” are assigned. In one embodiment, there is one temperature sensor; although in other embodiments, additional temperature and humidity sensors may be utilized. At 805, time variables from the user's “personal tuning strategy” are assigned to control the periods of time that the system should pause by delaying the execution of additional code instructions in order to control the following: the period of time to delay before checking the temperature again; the period of time that an HE should operate; and the period of time after an HE has operated that will allow the HE to return to the ambient temperature. At 806, variables for the power levels according to the user's personal tuning strategy stetting are assigned. In an embodiment, the personal tuning strategy levels for the duty cycle (voltage drawn by an HE) is measured in 256 power levels, which go from 0—fully off, to 255—fully on. At 807, the CPU is initiated, and at 808, the HEs are initiated.

Now referring to FIG. 8B, at 809, the system gets the actual voltage being delivered by the system. At 810, the system checks the temperature reading from the temperature sensor and, if the temperature reading is in the neutral zone (as determined at 811), the system remains off by delaying the execution of the code (at 812) (for the amount of time defined at 805 in the flowchart illustrated in FIG. 8A) before checking (at 810) the temperature again. If/when the temperature is not in the neutral zone, at 813, the HE's switch is operated to turn on the next HE in the control sequence to the on state duty cycle level (defined in flowchart step 806 in the flowchart illustrated in FIG. 8A). The HE is left on at this power level as the system delays the execution of the code (814) for the amount of time defined in flowchart step 805 before, at 815, turning off the HE via its switch by setting it to the off state power level. The system then delays the execution of the code (816) for the amount of time defined in flowchart step 806 in order to allow the HE that was controlled to return to the ambient temperature. In this embodiment, after step 816, the code will repeat flowchart steps 809 through 816 for each of the four (4) HEs controlled by the algorithm in the sequence for this embodiment. Also in this embodiment, when step 816 is reached for the last of the four (4) HEs in the control sequence, the system will repeat in a “loop” which is native to the CPU processing method starting with flowchart step 809 for the first HE.

FIGS. 9A and 9B are research diagrams, 910 and 920, respectively, illustrating various scientific aspects on which embodiments described herein are based.

With reference to FIG. 9A, thermoreceptors are cutaneous temperature-sensitive neurons. Thermoreceptors in mammals create seven discrete sensations 911. These sensations are brought on by exposure to various temperatures 912, which are shown on the x-axis in degrees Celsius (° C.). The sensations are experienced as a result of the firing of thermoreceptors at various rates 913, which are shown on the y-axis as impulses per second.

There are four types of thermoreceptor nerve fibers 914: cold-pain; cold receptor; warmth receptor, and heat-pain. Each of the thermoreceptor fiber types have limited firing capabilities, represented in this figure as solid and dashed lines 915 corresponding with the seven discrete sensations 911. The fiber types do not create sensation when they are not being exposed to their related temperature ranges. For example, a cold stimulus of 10° C. applied to a warmth receptor will not create a firing response in the warmth receptor.

The seven discrete sensations 911 and their approximate related temperatures 912 are: freezing cold, a painful sensation brought on by exposure to temperatures near freezing (5-12° C.) which could cause hypothermia and death; cold, an uncomfortable sensation brought on by very low temperatures (13-22° C.); cool, a mild sensation brought on by somewhat lower temperatures (23-30° C.) than the indifferent range; indifferent, a neutral (basically unnoticeable) sensation experienced in mild temperatures (31-36° C.); warm, a mild sensation brought on by temperatures somewhat higher (37-43° C.) than the indifferent range; hot, an uncomfortable sensation brought on by very high temperatures (44-51° C.); and burning hot, a painful sensation brought on by exposures to temperatures with the potential for causing burns or hyperthermia (51-60° C.).

FIG. 9A shows that, as temperature increases along the x-axis 912, the firing rate of cold pain fibers shown on the y-axis 913 decreases starting at 5° C. until the cold pain fibers no longer fire, at 15° C. At about 7° C., the cold receptors are activated. The cold receptors fire until 43° C., reaching their peak firing rate at about 25° C. Warmth receptors begin firing at 30° C. and continue firing until about 50° C., reaching their peak firing rate at about 42° C. The heat pain fibers activate near 45° C. and remain active until around 55° C.

FIG. 9B depicts thermoreceptors adapting over time to a constant thermal stimulus. In this figure, the y-axis has two sections, the top section 921 shows the frequency of receptor firing for a typical warm receptor 921a and cold receptor 921b. The frequency is represented by the proximity of the vertical lines 921c to one another along the x-axis: closer lines are high frequency, lines spaced farther apart are lower frequency. The bottom section 922 shows how temperature was applied at two levels, temperature level T1 922a and temperature level T2 922b. The x-axis represents time 923.

The upper section 921 of the drawing shows that during exposure to temperature level T1 922a, the warm receptor 921a and cold receptor 921b are firing at a similar frequency. When the receptors are exposed to temperature level T2 922b, the warm receptor 921a fires at a high frequency and the cold receptor 921b fires at a very low frequency. This would create a warm sensation. The frequency of the warm receptor firing 921a is greatest during the initial exposure 921d to temperature level T2 922b, then adapts over time 921e.

When the temperature level returns to T1, the cold receptor 921b fires at a high frequency and the warm receptor 921c fires at a very low frequency. During this period, the frequency of the cold receptor firing is greatest during the initial exposure 921f and adapts over time 921g. Ultimately, the thermoreceptors will fully adapt to the temperature level and return to the firing frequency shown in the section of the figure on the left of the x axis 922a.

FIG. 9B illustrates that, when first exposed to a sudden change in temperature, thermoreceptors fire at a high rate of frequency. But, as they continue to sense the same thermal stimulus over time, the same thermoreceptors will adapt and fire at a lower rate of frequency. Research has shown that this adaptation of thermoreceptors is significant after 10 seconds. The result of these findings is that a person will sense the temperature more strongly as it is changing, since that change will create the greatest firing of thermoreceptors and minimize their adaptation.

Other features of the systems and apparatuses described herein include the following: the system could have an algorithm component and hardware such as a buzzer for warning users about unsafe operation; the UHF is offered with DuPont Nomex fabric mesh, which is fire-resistant—using Nomex UHF heat exchangers could provide a greater level of safety for the user; in the heating shirt embodiment, the heat exchangers are UHF, but they could also be thermoelectric modules (TEMs) as a warming-only or a warming-cooling system where the polarity on the TEMs could be switched to change the direction of the heat exchange; in the cooling shirt embodiment, TEMs can have heat exchange reversed by changing the polarity of the current going to the TEMs, such as could be done with a dual pole dual throw (DPDT) switch; the systems could be powered via a cord and outlet, solar power or kinetic energy; the systems could be managed via an application and wirelessly connected to networks and/or other hardware and/or software; there could be pre-set user tuning strategies; there could be an enclosure made of plastic or other material that is waterproof; there could be switches on the enclosure or pouch for user-tunable parameters and power; the pouch or enclosure could be worn on parts of the body other than the waist; the pouch or enclosure could be attached to other parts of the garment, such as a sleeve; the system could be embedded in medical equipment, personal protective equipment, body armor, furniture and vehicle seating; other sensors could be employed (for example, a daylight sensor could adjust for users' solar heat gain by changing the level of system-delivered sensible heat or cooling; a humidity sensor could be used); temperature sensors could be located at each heat exchanger; the wire ribbon could be terminated at a connector plug and a matching connector receptacle could be put on the circuit board; the plug could be detached from the receptacle so the garment could be washed without the control board attached; the fans could turn off when the TEMs are off (the temperature is in the neutral zone); and the embodiments could be used by mammals other than humans. This should be done when the heat is removed from the TEM or after a delay. This would ensure that the TEMs are at the correct operating temperature when the TEMs are used again.

Embodiments described herein provide several advantages. The overall system is lightweight and compact enough to be worn by a typical adult. Because the system heats and cools the skin through conductance, there is no need for liquid or air to act as a heat transfer medium, which simplifies manufacturing and maintenance. Since the system directly addresses dermatomes, the system does not require operation in an enclosed comfort envelope (such as a sealed suit, vest or jacket). This means the system can be used as part of a base layer garment, enabling the user to be able to move their arms and torso freely. The system does not attempt to create comfort by cooling the whole body, which requires removing or delivering a significant amount of heat. This lowers the cost and simplifies operation, design and manufacturing. The system does not use heat exchangers on an “all on” basis over multiple dermatomes, so the design is energy efficient, meaning that smaller batteries can be used, thereby reducing weight and cost. The heat exchangers do not stay on for long periods of time, past the point of sensory adaptation, further increasing energy efficiency. The system is designed to provide a significant change in temperature when the heat exchangers operate, which is more effective at creating thermal comfort than maintaining a consistent temperature because it further avoids sensory adaptation.

It will be appreciated that the above figures and description provide exemplary, non-limiting configurations. Although the present invention has been described with reference to these exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as fall within the true spirit and scope of the invention.

1. A system for providing a sensation of warmth to a user, the system comprising:
a central processing unit (CPU) for running an algorithm that manages a personal tuning strategy of the user;
one or more temperature sensors connected to the CPU and operable to obtain a temperature reading of the user;
a plurality of transistor switches, for switching electronic signals, connected to the CPU;
a plurality of heat exchange elements capable of conducting sensible heat, each connected to and corresponding to a respective one of the plurality of transistor switches;
a matrix for associating the plurality of heat exchange elements to a plurality of dermatomes of the user, wherein one of the plurality of heat exchange elements corresponds to a respective one or more of the plurality of dermatomes of the user;
wherein the CPU obtains a temperature reading from the one or more temperature sensors and, if the temperature reading is not within limits defined by the personal tuning strategy, the CPU implements the algorithm of:
turning on each transistor switch, of the plurality of transistor switches, in sequence to deliver power to the corresponding heat exchange element, of the plurality of heat exchange elements in the heat exchanger matrix, for a predetermined period of time to provide a heating thermal sensation to the user at the respective one or more of the plurality of dermatomes; and
turning off each transistor switch in sequence;
wherein the CPU operates to implement the algorithm until a new temperature reading is within the limits defined by the personal tuning strategy.

2. The system of claim 1, further comprising one or more humidity sensors connected to the CPU and operable to obtain a humidity reading of the user;
wherein the CPU implements the algorithm when a humidity reading from the one or more humidity sensors is not within limits defined by the personal tuning strategy, and stops implementing the algorithm when a new humidity reading is within the limits defined by the personal tuning strategy.

3. The system of claim 1, wherein the CPU further operates to control voltage and amperage delivered through the plurality of transistor switches to match a target voltage setting based from the personal tuning strategy.

4. The system of claim 1, wherein the plurality of heat exchange elements are placed near or adjacent to the user, via the matrix, to correspond with alternating dermatomes, wherein each of the plurality of heat exchange elements is sized to simultaneously address two dermatomes.

5. The system of claim 1, wherein the plurality of heat exchange elements and the matrix are incorporated in a garment worn by the user.

6. The system of claim 1, wherein the CPU and the plurality of transistor switches are part of a circuit board assembly connectable to a garment worn by the user.

7. The system of claim 1, wherein the personal tuning strategy is inputted to the CPU via an application.

8. A system for providing a sensation of coolness to a user, the system comprising:
a central processing unit (CPU) for running an algorithm that manages a personal tuning strategy of the user;
one or more temperature sensors connected to the CPU and operable to obtain a temperature reading of the user;
a plurality of transistor switches, for switching electronic signals, connected to the CPU;
a plurality of heat stack elements, each connected to and corresponding to a respective one of the plurality of transistor switches, wherein each of the plurality of heat stack elements is comprised of: a thermoelectric (TEM) heat exchanger capable of conducting sensible cooling, a heat sink, and a fan;
a matrix for associating the plurality of heat stack elements to a plurality of dermatomes of the user, wherein one of the plurality of heat stack elements corresponds to a respective one or more of the plurality of dermatomes of the user;
wherein the CPU obtains a temperature reading from the one or more temperature sensors and, if the temperature reading is not within limits defined by the personal tuning strategy, the CPU implements the algorithm of:
turning on each transistor switch, of the plurality of transistor switches, in sequence to deliver power to the corresponding heat stack element, of the plurality of heat stack elements, for a predetermined period of time to provide a cooling sensation to the user at the respective one or more of the plurality of dermatomes; and
turning off each transistor switch in sequence;
wherein the CPU operates to implement the algorithm until a new temperature reading is within the limits defined by the personal tuning strategy.

9. The system of claim 8, further comprising one or more humidity sensors connected to the CPU and operable to obtain a humidity reading of the user;
wherein the CPU implements the algorithm when a humidity reading from the plurality of humidity sensors is not within limits defined by the personal tuning strategy, and stops implementing the algorithm when a new humidity reading is within the limits defined by the personal tuning strategy.

10. The system of claim 8, wherein the CPU further operates to control voltage and amperage delivered through the plurality of transistor switches to match a target voltage setting based from the personal tuning strategy.

11. The system of claim 8, wherein the plurality of heat stack elements and the matrix are incorporated in a garment worn by the user.

12. The system of claim 8, wherein the CPU and the plurality of transistor switches are part of a circuit board assembly connectable to a garment worn by the user.

13. The system of claim 8, wherein the personal tuning strategy is inputted to the CPU via an application.

14. A warming apparatus, comprising:
a fabric channel laminated to a matching sheet of adhesive;
a layer of garment adhesive laminated to a garment and bonded to the sheet of fabric channel adhesive;
a plurality of heat exchange elements capable of conducting sensible heat;
a plurality of pieces of reflective insulation, each piece corresponding to one of the plurality of heat exchange elements;
a matrix for associating the plurality of heat exchange elements to a plurality of dermatomes of a user;
wherein the plurality of heat exchange elements are wired to a circuit board assembly comprising a central processing unit (CPU) that implements an algorithm of (i) turning on one of the plurality of heat exchange elements in sequence for a predetermined period of time to provide a heating thermal sensation to the user at the related dermatomes based on a sensed temperature reading not within limits defined by the user, (ii) turning off said heat exchange elements in sequence, and (iii) stopping the sequence when a new temperature reading is within the limits defined by the user.
wherein the plurality of heat exchange elements and plurality of pieces of reflective insulation are located between the garment adhesive and the fabric channel adhesive.

15. The apparatus of claim 15, further comprising one or more temperature sensors, wherein the CPU obtains a temperature reading from the one or more temperature sensors and determines if the temperature reading is within the limits defined by user.

16. The apparatus of claim 15, wherein one or more of the garment adhesive and the fabric channel adhesive are a thin thermoplastic polyurethane (TPU) adhesive.

17. The apparatus of claim 15, further comprising a pouch for containing the circuit board assembly.

18. A cooling apparatus, comprising:
a fabric channel laminated to a matching sheet of adhesive;
a layer of garment adhesive laminated to a garment and bonded to the sheet of fabric channel adhesive,
a plurality of heat stack elements, each heat stack element comprising: a thermoelectric (TEM) heat exchanger capable of conducting sensible cooling, a heat sink, and a fan;
a matrix for associating the plurality of heat stack elements to a plurality of dermatomes to a plurality of dermatomes of a user;
wherein the plurality of heat stack elements are wired to a circuit board assembly comprising a central processing unit (CPU) that implements an algorithm of (i) turning on one of the plurality of heat stack elements in sequence for a predetermined period of time to provide a cooling sensation to the user at the related dermatome based on a sensed temperature reading not within limits defined by a user, (ii) turning off said heat stack elements in sequence, and (iii) stopping the sequence when a new temperature reading is within the limits defined by the user;
wherein the plurality of heat stack elements are located between the garment adhesive and the fabric channel adhesive to allow fins of the heat sinks to pass through the fabric channel adhesive and the fabric channel.

19. The apparatus of claim 18, further comprising one or more temperature sensors, wherein the CPU obtains a temperature reading from the one or more temperature sensors and determines if the temperature reading is within the limits defined by user.

20. The apparatus of claim 18, wherein one or more of the garment adhesive and the fabric channel adhesive are a thin thermoplastic polyurethane (TPU) adhesive.
